(19) United States
(12) Patent Application Publication
(10) Pub. No.: US 2008/0058399 A1
(43) Pub. Date: Mar. 6, 2008

(54) Pharmaceutical Compositions

(75) Inventors: Bertil Abrahamsson, Molndal (SE); Jonas Odman, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/850,049

(22) Filed: Sep. 5, 2007

Related U.S. Application Data
(60) Provisional application No. 60/842,432, filed on Sep. 5, 2006.

(51) Int. Cl. A61K 31/41; A61P 43/00

(52) U.S. Cl. 514/381

(57) ABSTRACT

The present invention provides pharmaceutical compositions comprising as an active ingredient candesartan or candesartan cilexetil, compositions exhibiting a relative bioavailability, measured as area under the curve, of more than 1.5.

### CROSS-REFERENCE TO RELATED APPLICATIONS

This application claims priority to U.S. provisional application Ser. No. 60/842,432 filed Sep. 5, 2006, which is incorporated herein by reference in its entirety.

### FIELD OF THE INVENTION

The present invention is directed, in part, to pharmaceutical compositions comprising candesartan cilexetil, methods for preparing such compositions, methods of using such compositions in the manufacture of a medicament for use in therapy, and methods of treating a patient by administering such a composition.

### BACKGROUND OF THE INVENTION

Candesartan cilexetil is a prodrug of candesartan—a compound that inhibits binding of angiotensin II to the AT₁-receptor. Candesartan cilexetil is sparingly soluble in water (<0.05 μg/ml).

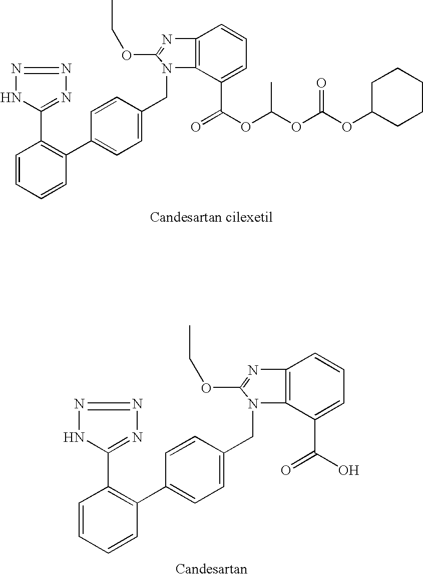

The bioavailability of a pharmaceutically active ingredient depends on several factors, a major factor being the solubility of the active ingredient in water. The rate limiting step for the absorption of a sparingly water-soluble pharmaceutical active ingredient (that is one which requires more than 5000 g of water to dissolve 1 g of active ingredient) administered in solid form is the dissolution of the active ingredient in water.

The present invention provides for pharmaceutical compositions comprising as an active ingredient candesartan or candesartan cilexetil, the composition exhibiting a relative bioavailability, measured as area under the curve (AUC), of more than 1.5.

### SUMMARY OF THE INVENTION

The present invention provides pharmaceutical compositions comprising as an active ingredient candesartan or candesartan cilexetil, the composition exhibiting a relative bioavailability, measured as area under the curve (AUC), of more than 1.5. In some embodiments, the active ingredient is in a substantially non-crystalline form. In some embodiments, the composition further comprises a solubilizer. In some embodiments, the relative bioavailability is more than 2.

### DESCRIPTION OF EMBODIMENTS

The present invention provides pharmaceutical compositions comprising as an active ingredient candesartan or candesartan cilexetil, the composition exhibiting a relative bioavailability, measured as area under the curve (AUC), of more than 1.5.

According to one aspect of the invention, the active ingredient is in a substantially non-crystalline form.

According to one aspect of the invention, the composition further comprises a solubilizer.

According to one aspect of the invention, the relative bioavailability of the composition is more than 2.

The pharmaceutical composition is solid at 25° C. The non-crystalline state of candesartan cilexitil can be obtained by dispersion of the drug in a matrix comprising one or more solubilizers and/or one or more water-soluble polymers, or by a specifically designed process for precipitation of pure drug in the desired state.

According to an aspect of the invention the pharmaceutical composition comprises active ingredient and a solubilizer.

According to an aspect of the invention, the composition comprises active ingredient, a solubilizer, a disintegrant, a binder, and a lubricant.

According to an aspect of the invention, the composition comprises candesartan cilexetil, propylene oxide, starch, microcrystalline cellulose and sodium stearyl fumarate.

A solubilizer is a compound that enhances the solubility of another compound in water. Suitable solubilizers include: Surface active agents. Surface active agents can be non-ionic, anionic, cationic or zwitterionic surfactants. Suitable non-ionic surfactants include polyoxyethylene sorbitan fatty acid esters, sorbitan fatty acid esters, polyoxyethylene alkyl ethers or sucrose esters. Suitable anionic surfactants include sodium dodecyl sulphate, sodium 1,4-bis(2-ethylhexyl)sulphosuccinate or salts of fatty acids. Suitable cationic surfactants include alkyltrimethylammonium salts or dialkyldimethylammonium salts. Suitable zwitterionic surfactants include 3((3-cholamidopropyl)dimethylammonio)-1-propane sulphonate or dodecyl-N-betaine.

Suitable non-ionic surfactants with a hydrophilic predominance, having a hydrophilic-lipophilic balance of more than 12, include polyoxyethylenic esters of sorbitan or fatty acids (such as TWEEN 20 to 80), a polyoxyethylenic ether of a fatty alcohol (such as BRIJ 56, 58, 78, 96, 98, 99 or Cremophor) or a block-copolymer of ethylene oxide and propylene oxide (such as a POLOXAMER, for example PLURONIC F68 or F87).

Water-soluble polymers can also be used to obtain candesartan cilexetil in a non-crystalline state. Water-soluble polymers include: an alkylcellulose (such as methylcellulose), a hydroxyalkylcellulose (such as hydroxymethylcellulose, hydroxyethylcellulose, hydroxypropylcellulose or hydroxybutylcellulose), a hydroxyalkyl alkylcelluloses (such as hydroxyethyl methylcellulose or hydroxypropyl methylcellulose), a carboxyalkylcellulose (such as carboxymethylcellulose), an alkali metal salt of a carboxyalkylcellulose (such as sodium carboxymethylcellulose), a carboxyalkylalkyl-cellulose (such as carboxymethylethylcellulose), a carboxyalkylcellulose ester, a starch, a pectin (such as sodium carboxymethylamylopectine), a chitin derivate (such as chitosan), a polysaccharide (such as alginic acid or an alkali metal or ammonium salt thereof, a carrageenan, a galactomannan, tragacanth, agar-agar, gum arabicum, guar gum, xanthan gum or gelatin), a polyacrylic acid or a salt thereof (such as a polymethacrylic acid or a salt thereof, or a metacrylate copolymer), a polyvinylalcohol, a polyvinylpyrrolidone (such as a co-polymer of polyvinylpyrrolidone with vinyl acetate and cross-linked polyvinylpyrrolidone) or a polyalkylene oxide (such as polyethylene oxide, polypropylene oxide or a co-polymer of ethylene oxide and propylene oxide), or any subgroup thereof. In one aspect of the invention, the water-soluble polymer is polyvinyl pyrrolidone or a co-polymer of ethylene oxide and propylene oxide (a poloxamer).

The ratio of candesartan cilexetil to said water-soluble polymer may be 1:9 to about 9:1, for instance 1:9 to 3:1. A mixture of two or more water-soluble polymers may also be used in a composition according to the invention.

Solid dosage forms include tablets (including, for example, immediate release tablets, extended release tablets, coated tablets, gel coated tablets and enteric coated tablets), capsules (e.g. soft gelatin capsules and hard gelatin capsules), pellets or particles. Known excipients (e.g. bulking agents, binders, disintegrants, lubricants, glidants, surfactants, can be blended with these systems given above to provide the desired dosage form.

Solid dispersions of drug and carrier material can be prepared by a solvent method wherein the drug is dissolved before mixing with carrier material and other excipients. Alternatively, the carrier material could be dissolved in the drug solution before mixing with other excipients.

Alternatively, solid dispersions can be prepared by grinding candesartan cilexetil together with water-soluble carrier material.

Solid dispersions may be prepared by the method of dissolution followed by evaporation, melting followed by solidification or a combination of these methods.

The melting method requires that candesartan cilexetil and the water-soluble carrier are miscible in the molten state. When the solvent method is used, both candesartan cilexetil and the water-soluble carrier are dissolved in a mutual solvent which is removed (such as by evaporation under reduced pressure, spray-drying, freeze-drying, supercritical crystallization or other similar technique). Solid dispersions prepared by the solvent method are sometimes termed co-precipitates or co-evaporates. In the melting-solvent method the dissolved candesartan cilexetil is added to the melted carrier.

The solvent method comprises dissolving candesartan cilexetil in a volatile organic solvent containing at least one hydrophilic polymer carrier and evaporating the solvent to dryness to form a co-evaporate of candesartan cilexetil and hydrophilic polymer carrier(s). The dissolution rate of the resulting co-evaporate can be further increased by adding surface active agent(s) to the organic solvent either before or after evaporation. Other additives may also be added such as a disintegrant.

Suitable solvents include oxygenated solvents (such as an alcohol, ether or ketone {for example ethanol, i-propanol, tetrahydrofuran, i-propyl ether, tetrahydropyran, acetone or methyl ethyl ketone) or chlorinated solvents (such as methylene chloride, chloroform or mixtures in various proportions of these same solvents).

The melting method comprises a) melting at least one of the components and dispersing the other(s) in this melt or b) melting of all components. At least one component may be a hydrophilic polymer. Other additives may also be added such as a surfactant or a disintegrant.

Small non-crystalline particles of candesartan cilexetil may be prepared by a spontaneous emulsion solvent diffusion method, comprising the dissolution of candesartan cilexetil in an organic water-miscible solvent or a mixture of organic water-miscible solvents (such as methanol, ethanol, i-propanol, acetonitrile, acetone or dimethylsulphoxide) followed by precipitation of candesartan cilexetil by mixing the organic solvent(s) with an aqueous phase. To prevent agglomeration, the aqueous phase preferably contains a hydrophilic polymer.

The presence of an additive in the aqueous phase and/or in the organic solvent can influence the rate of precipitation, giving rise to different drug particle sizes and morphologic states. Suitable additives include a polymer (for example polyethylene glycol, polyvinyl pyrrolidone, methylcellulose or hydroxymethylcellulose), a salt (such as sodium chloride, calcium chloride or aluminum chloride), a viscosity enhancing agent (such as gelatin, acacia) or a co-solvent (such as glycerol or propylene glycol).

The small particles can be collected by various methods, such as: centrifugation or ultracentrifugation, filtration, reverse osmosis followed by evaporation, evaporation of the solvent by heating and/or vacuum, freeze-drying, spray-drying, fluidized-bed drying or a combination of any of the above.

The solid state of the small particles may be of amorphous character or partially crystalline when the precipitation is rapid, that is rapid diffusion of the organic solvent in the aqueous phase (for example due to similarities in the dielectricity constants of the organic solvent and water). If the diffusion process is slower, candesartan cilexetil may precipitate as crystals. The hydrophobic nature of candesartan cilexetil in relation to the aqueous solvent is also important for which sort of particles that are created, that is particle size and morphologic state.

In order that the invention disclosed herein may be more efficiently understood, examples are provided below. It should be understood that these examples are for illustrative purposes only and are not to be construed as limiting the invention in any manner.

### EXAMPLES

In this section, the materials, the preparation techniques and the analytical methods used in the following examples are described.

Where used, candesartan cilexetil (abbreviated to c.c.), CREMOPHOR RH40™ (a polyoxyl 40 hydrogenated castor oil), polyethylene glycol 6000, polyvinyl pyrrolidone K90, maize starch, aluminum silicate, mannitol, AVICEL™ (a microcrystalline cellulose), cross-linked polyvinyl pyrrolidone, magnesium stearate, sodium stearyl fumarate (PRUV) and ethanol (95%) were supplied by Astra AB, Poloxamer 188 (PLURONIC F68™) was supplied by BASF and polysorbate 20 (TWEEN 20™) was supplied by Acros.

The medium used for dissolution studies was 500 ml of 0.2% polysorbate 20 in 0.05 M phosphate buffer pH 6.5 with a paddle speed of 75 rpm (USP dissolution apparatus II). In all dissolution tests, 8 mg of candesartan cilexetil was added to the medium (except where other is stated). After extraction with 60% (v/v) acetonitrile in water (filter pore size 0.45 μm), the amount of candesartan cilexetil was determined by liquid chromatography and UV-detection at 254 nm.

Particles were studied in a scanning electron microscopy (JEOL JSM-5400). The dispersions and particles were studied by FT-Raman (Perkin Elmer 2000).

### Example 1

This Example illustrates the preparation of a solid dispersion of candesartan cilexetil in Poloxamer 188 and prepared by a melting-solidification method.

Part A

| | |
|---|---|
| Candesartan cilexetil | 10-20% |
| Poloxamer 188 | 80-90% |

A mixture of Candesartan cilexetil in crystalline powder form and Poloxamer 188 was heated at about 70° C. for about 5 minutes (that is above the melting temperature of the polymer but below the melting of the active compound) and the mixture was allowed to cool naturally. After solidification at ambient temperature the dispersion was milled using a Stomacher. The resulting particles were sieved through a 0.7 mm sieve.

The active compound in the dispersion was found by Raman analysis to be crystalline.

| | Drug | Drug | Drug | |
|---|---|---|---|---|
| | dissolved | dissolved | dissolved | |
| | after | after 10 | after 15 | Drug dissolved |
| | 5 minutes | minutes | minutes | after 20 minutes |
| c.c. | 24 | 43 | 54 | 61 |
| Physical mixture | 28 | 44 | 55 | 61 |
| of 20% c.c. and | | | | |
| Poloxamer 188 | | | | |
| 10% c.c. in | 57 | 74 | 81 | 86 |
| Poloxamer 188 | | | | |
| 20% c.c. in | 48 | 68 | 79 | 84 |
| Poloxamer 188 | | | | |

Part B: Formation of a Tablet

The milled melt from Part A was mixed with an immediate release granulate (primarily containing microcrystalline cellulose and maize starch) in a ratio of 1:2 to 1:5 (Part A to granule) and tablets were made by compression of the resulting mixture. The tablet disintegrated in an aqueous solution within 10 minutes at 37° C.

| | Drug | Drug | Drug | |
|---|---|---|---|---|
| | dissolved | dissolved | dissolved | |
| | after | after 10 | after 15 | Drug dissolved |
| | 5 minutes | minutes | minutes | after 20 minutes |
| candesartan | 24 | 43 | 54 | 61 |
| cilexetil (c.c.) | | | | |
| Tablet with solid | 39 | 58 | 68 | 75 |
| dispersion of 20% | | | | |
| c.c. in poloxamer | | | | |
| 188* | | | | |

*The tablet contained 7.7 mg c.c.

### Example 2

This Example illustrates the preparation of a solid dispersion of candesartan cilexetil in polyethylene glycol 6000 prepared by a melting—solidification method.

Part A

| | |
|---|---|
| Candesartan cilexetil | 10-20% |
| Polyethylene glycol 6000 (PEG 6000) | 80-90% |

A physical mixture of Candesartan cilexetil and PEG 6000 was heated at about 70° C. (that is, above the melting temperature of the polymer, but well below the melting of the active compound) for about 5 minutes and the mixture was allowed to cool naturally. After solidification at ambient temperature the dispersion was milled using a Stomacher. The resulting particles were sieved through a 0.7 mm sieve.

The drug in the dispersion was found with Raman analysis to be crystalline.

| | Drug | Drug | Drug | |
|---|---|---|---|---|
| | dissolved | dissolved | dissolved | |
| | after | after 10 | after 15 | Drug dissolved |
| | 5 minutes | minutes | minutes | after 20 minutes |
| c.c. | 24 | 43 | 54 | 61 |
| 10% c.c. in | 63 | 78 | 84 | 88 |
| PEG 6000 | | | | |
| 20% c.c. in | 41 | 58 | 68 | 78 |
| PEG 6000 | | | | |
| Physical mixture | 28 | 45 | 55 | 63 |
| of 20% c.c. and | | | | |
| PEG 6000 | | | | |

Part B: Formation of a Tablet

In a method similar to that of Part B of Example 1, the milled dispersion was mixed with an immediate release granulate and tablets were made. The tablet disintegrates in an aqueous solution within 10 minutes at 37° C.

| | Drug | Drug | Drug | |
|---|---|---|---|---|
| | dissolved | dissolved | dissolved | |
| | after | after 10 | after 15 | Drug dissolved |
| | 5 minutes | minutes | minutes | after 20 minutes |
| candesartan | 24 | 43 | 54 | 61 |
| cilexetil (c.c.) | | | | |
| Tablet with solid | 54 | 70 | 78 | 84 |
| dispersion of | | | | |
| 10% c.c. in PEG | | | | |
| 6000* | | | | |
| Tablet with solid | 53 | 71 | 79 | 85 |
| dispersion of | | | | |
| 20% c.c. in PEG | | | | |
| 6000** | | | | |

*The tablet contained 7.5 mg c.c.
**The tablet contained 8.1 mg c.c.

### Example 3

This Example illustrates the preparation of a tablet comprising a solid dispersion of candesartan cilexetil in polyvinyl pyrrolidone K90 and prepared by the dissolution-evaporation method.

| | |
|---|---|
| Candesartan cilexetil | 45% |
| Polyvinyl pyrrolidone K90 (PVP K90) | 55% |

Candesartan cilexetil was dissolved (45 mg/ml), together with 7% (w/w) PVP K90, in ethanol. The resulting solution was used as a granulating liquid with a powder bed of microcrystalline cellulose, mannitol and aluminium silicate to form an immediate release granulate. After drying at 50° C. the resulting mixture was compressed to form tablets. The tablets disintegrated in an aqueous solution within 10 minutes at 37° C.

The drug in the dispersion was found by Raman analysis to be amorphous.

| | Drug | Drug | Drug | |
|---|---|---|---|---|
| | dissolved | dissolved | dissolved | |
| | after | after 10 | after | Drug dissolved |
| | 5 minutes | minutes | 15 minutes | after 20 minutes |
| c.c. | 24 | 43 | 54 | 61 |
| *Tablet with | 53 | 69 | — | 84 |
| solid dispersion | | | | |
| of 45% c.c. in | | | | |
| PVP K90 | | | | |

*The tablet contained 7.8 mg.

### Example 4

This Example illustrates the preparation of a solid dispersion of candesartan cilexetil in polyvinyl pyrrolidone K90 and prepared by the dissolution-evaporation method with the addition of a surfactant.

| | |
|---|---|
| Candesartan cilexetil | 31% or 21% |
| PVP K90 | 38% or 26% |
| CREMOPHOR RH40 | 31% or 53% |

The active compound was dissolved (45 mg/ml), together with 7% (w/w) PVP K90 and CREMOPHOR RH40 (45 or 112.5 mg/ml) in ethanol. Tablets were formed using the methodology of Example 3. A tablet disintegrated in an aqueous solution within 10 minutes at 37° C.

The drug in the dispersion was found by Raman analysis to be partially crystalline.

| | Drug | Drug | Drug | |
|---|---|---|---|---|
| | dissolved | dissolved | dissolved | |
| | after | after 10 | after 15 | Drug dissolved |
| | 5 minutes | minutes | minutes | after 20 minutes |
| c.c. | 24 | 43 | 54 | 61 |
| Tablet with solid | 56 | 68 | — | 83 |
| dispersion of | | | | |
| 45% c.c. in PVP | | | | |
| K90 and with | | | | |
| Cremophor | | | | |
| RH40 (1 mg/mg | | | | |
| c.c.)* | | | | |
| Tablet with solid | 41 | 82 | — | 93 |
| dispersion of | | | | |
| 45% c.c. in PVP | | | | |
| K90 and with | | | | |
| Cremophor | | | | |
| RH40 | | | | |
| (2.5 mg/mg | | | | |
| c.c.)** | | | | |

*The tablet contained 8.0 mg
**The tablet contained 7.9 mg

### Example 5

This Example illustrates the preparation of non-crystalline candesartan cilexetil particles having a diameter of 250-350 nm.

The active compound was dissolved (10 mg/ml) in 30 ml ethanol. This solution was slowly added to aqueous solution of 1.5% (w/w) PVP K90 (125 ml) and particles formed. The particle suspension was washed with water during centrifugation and the water volume was reduced to about 2-3 ml. The suspension was mixed with an equal volume of 10% (w/w) PVP in water and used as a granulating liquid (5% PVP K90) with a premixed powder blend of microcrystalline cellulose, mannitol and primojel to form an immediate release granulate which was compressed to form tablets. A tablet disintegrated in an aqueous solution within 10 minutes of contact with water at 37° C.

The particles formed were found by Raman analysis to be totally amorphous.

| | | | Drug | Drug |
|---|---|---|---|---|
| | | | dissolved | dissolved |
| | Drug dissolved | Drug dissolved | after 15 | after 20 |
| | after 5 minutes | after 10 minutes | minutes | minutes |
| c.c. | 24 | 43 | 54 | 61 |
| Tablet with | 75 | 90 | 96 | 98 |
| small | | | | |
| amorphous | | | | |
| particles* | | | | |

*The tablet contained 5.4 mg drug.

### Example 6

This Example illustrates the preparation of amorphous candesartan cilexetil particles having a diameter of 1-3 μm.

The active compound was dissolved (60 mg/ml) in 10% PVP K90 (w/w) in ethanol. This solution was slowly added to an equal volume of an aqueous solution of 1.5% (w/w) PVP K90 and particles formed. The particle suspension was used as a granulating liquid (˜5.3% (w/w) PVP K90) with a premixed powder blend of microcrystalline cellulose, mannitol and primojel to form an immediate release granulate which was compressed to form tablets. A tablet disintegrated in an aqueous solution within 10 minutes at 37° C.

The particles were found by Raman analysis to be mainly amorphous (some crystalline content).

| | Drug | Drug | Drug | |
|---|---|---|---|---|
| | dissolved | dissolved | dissolved | |
| | after 5 | after 10 | after 15 | Drug dissolved |
| | minutes | minutes | minutes | after 20 minutes |
| candesartan | 24 | 43 | 54 | 61 |
| cilexetil (c.c.) | | | | |
| Tablet with large | 63 | 83 | — | 92 |
| amorphous | | | | |
| particles* | | | | |

*The tablet contained 6.6 mg drug.

### Example 7

The bioavailability for solid dispersions (poloxamer 188) and for small amorphous particles were studied in rats. The dispersions were per-orally administrated as powders and the particles were given in suspensions.

The relative bioavailability in rat for candesartan cilexetil given as a suspension is 19% and given in solution is 50%.

The suspension with small amorphous drug particles had a relative bioavailability of 40%. The relative bioavailability for solid dispersion of candesartan cilexetil in poloxamer 188 was 25%.

### Example 8

The bioavailability was determined in human volunteers for one tablet (A) containing candesartan cilexitil mainly in non-crystalline form, one tablet (B) containing mainly crystalline drug dispersed in a tablet matrix consisting of a rapidly dissolving hydrophilic polymer in relation to a standard tablet (C) including mainly crystalline drug. All tablets contained 32 mg candersartan cilexitil. The study was performed according to a cross-over design and each tablet was administrated as a single dose to 15 healthy volunteers. The relative bioavailability of tablet A and B in relation to tablet C was determined from the area under the curve (AUC) of the candersartan plasma concentration-time curves.

The mean AUC-ratio for tablet A in relation to tablet C was 2.55, (i.e., the bioavailability was more than doubled for tablet A compared to the standard tablet). The corresponding AUC-ratio for tablet B was 1.24 and no statistically significant (p>0.05) was detected for this tablet compared to the reference.

Tablet A composition (Ingredients for 2000 tablets)

| | |
|---|---|
| Candesartan cilexetil | 16 g |
| Maize starch | 107 g |
| Microcrystalline cellulose | 288 g |
| Poloxamer 188 | 64 g |
| Sodium stearyl fumarate | 5 g |
| Water, purified* | q.s. |

*Used in the manufacturing process, but removed during drying.

Tablet B composition (Ingredients for 2000 tablets)

| | |
|---|---|
| Candesartan cilexetil | 16 g |
| Aliminium silicate | 20 g |
| Ethanol 95% (v/v)* | q.s. |
| Magnesium stearate | 4.6 g |
| Mannitol | 40 g |
| Microcrystalline cellulose | 256 g |
| Polyoxyl 40 hydrogenated castor oil | 40 g |
| Polyvinylpyrrolidone, cross-linked | 64 g |
| Polyvinlypyrrolidone, K90 | 20 g |

*Used in the manufacturing process, but removed during drying.

Tablet C Composition
Tablet C is a commercially available 8 mg candesartan cilexetil sold under the name of ATACAND™ by AstraZeneca AB.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, and the like) cited in the present application is incorporated herein by reference in its entirety. While particular embodiments of the subject invention have been described, it will be obvious to those skilled in the art that various changes and modifications of the subject invention can be made without departing from the spirit and scope of the invention. It is intended to cover, in the appended claims, all such modifications that are within the scope of this invention.

What is claimed is:

1. A pharmaceutical composition comprising as an active ingredient candesartan or candesartan cilexetil, the composition exhibiting a relative bioavailability, measured as area under the curve (AUC), of more than 1.5.

2. A pharmaceutical composition according to claim 1 wherein the active ingredient is in a substantially non-crystalline form.

3. A pharmaceutical composition according to claim 1 wherein the composition further comprises a solubilizer.

4. A pharmaceutical composition according to claim 2 wherein the composition further comprises a solubilizer.

5. A pharmaceutical composition according to claim 1 wherein the relative bioavailability is more than 2.

6. A pharmaceutical composition according to claim 2 wherein the relative bioavailability is more than 2.

7. A pharmaceutical composition according to claim 3 wherein the relative bioavailability is more than 2.

8. A pharmaceutical composition according to claim 4 wherein the relative bioavailability is more than 2.
